(19) United States
(12) Patent Application Publication
(10) Pub. No.: US 2022/0233608 A1
(43) Pub. Date: Jul. 28, 2022

(54) NATURAL FEED COMPOSITION DERIVED FROM FRESH WATER ALGAL CULTURES FOR THE PROMOTION OF ANIMAL GROWTH

(71) Applicant: ZIVO Bioscience, Inc., Keego Harbor, MI (US)

(72) Inventors: Amy E. Steffek, Keego Harbor, MI (US); Andrew A. Dahl, Keego Harbor, MI (US); William P. Pfund, Keego Harbor, MI (US)

(73) Assignee: ZIVO Bioscience, Inc., Keego Harbor, MI (US)

(21) Appl. No.: 17/410,016

(22) Filed: Aug. 24, 2021

Related U.S. Application Data
(60) Provisional application No. 63/056,993, filed on Jul. 27, 2020.

(51) Int. Cl. A61K 35/74; A23K 50/75; A23K 10/12; A23K 10/18; A23K 20/163

(52) U.S. Cl. CPC A61K 35/74; A23K 50/75; A23K 20/163; A23K 10/18; A23K 10/12

(57) ABSTRACT

A method and compound for promoting growth in both animals and humans are disclosed. The disclosed method utilizes a compound derived from a lipopolysaccharide (LPS) of gram-negative bacteria. The compound itself is a natural product with no observed adverse environmental impact. The compound is combined with conventional feed, such as a corn-soy feed, for administration to animals, such as poultry. Human application is possible, as well. The combination of the disclosed inventive compound and conventional feed works through multiple growth-related pathways within, for example, in healthy birds to enhance growth performance while also priming the immune system to expedite response to a disease challenge should one arise. Data indicate that the dietary mixture of the inventive compound in conventional feed suggests that the biomass alters the signaling of multiple growth-related pathways.

### CROSS REFERENCE TO RELATED APPLICATION

This application is a U.S. Non-provisional patent application of U.S. Provisional Patent Application No. 63/056,993, entitled “Natural Feed Composition Derived from Fresh Water Algal Cultures for the Promotion of Animal Growth,” filed Jul. 27, 2021, which is herein incorporated by reference in its entirety for all purposes.

### TECHNICAL FIELD

The present disclosed inventive concept relates to animal feed. More particularly, the disclosed inventive concept relates to a natural feed composition derived from fresh water algal cultures for the promotion of animal growth. The disclosed inventive concept has particular application in the poultry industry but may also find applications beyond poultry to other animals. The disclosed inventive concept may also be beneficial to humans.

### BACKGROUND OF THE INVENTION

The commercial animal industry is under constant economic pressure to develop methods for raising healthy animals that maximize the growth rates while minimizing costs related to feeding and care. One such industry is the poultry industry which is facing dramatic increases in production demand. Poultry meat competes with pork as the world's most consumed meat. It is expected that world poultry production will need to meet an increase in demand of over 120% by the year 2050.

In response to this increasing demand for supply, poultry producers are constantly seeking ways to increase the efficiency with which birds, such as broilers, convert feed into body mass as a way of increasing profit margin by decreasing the amount of feed required to produce birds of a certain size (or to maximize the size of birds produced using a given amount of feed). For farms where diseases such as coccidiosis are a significant problem, the focus on growth performance improvement is often directed at controlling disease which adversely affects growth performance. This approach, however, does nothing to improve growth performance of healthy birds.

Improving growth performance of healthy birds may be accomplished by either (1) increasing availability of nutrients in feed (by increasing digestibility, for example), (2) altering the physiology of the birds such that they metabolize available nutrients more efficiently, or (3) by shifting energy utilization in the birds away from non-growth related processes to growth-related metabolic pathways.

Accordingly, it is desirable to develop a practical, natural, and cost-effective method of feeding animals to increase growth and to support overall animal health, thereby making the animal more resilient to disease.

### SUMMARY OF THE INVENTION

The disclosed inventive concept provides a natural compound for use as a feed ingredient to promote animal growth. The compound of the disclosed inventive concept is combined with conventional feed for administration to animals, such as poultry. Human application is possible, as well. The combination of the disclosed inventive compound and conventional feed works through unique pathways. For example, in healthy birds it serves to enhance growth performance while also priming the immune system (as disclosed in U.S. patent application Ser. No. 17/358,878 titled “Immune Priming to Accelerate/Enhance Immune Response Through Administration of Natural Immune Modulator,” incorporated by reference herein) to expedite response to a disease challenge should one arise. The disclosed inventive compound is a natural product and thus has no adverse environmental impact.

During the treatment period, the disclosed inventive compound or biomass comprising an algal culture is administered to the animal by way of poultry feed, drinking water, or both. Studies based on the use of animal feed stock including the disclosed inventive compound or biomass comprising an algal culture revealed improved growth in animals. It was found that the animals, particularly healthy birds, benefited significantly in terms of improved growth efficiency when fed the biomass of the disclosed inventive compound mixed with a conventional diet, such as a corn-soy diet. Data indicate that feeding healthy chickens (specifically, broiler chickens) a corn/soy diet supplemented with biomass, the inventive compound of algal culture, improves growth efficiency of the birds such that the Body Weight Gain (BWG) and Feed Conversion Ratio (FCR) are improved in a statistically significant manner compared to birds fed the same diet without algal culture biomass supplementation. It should be understood that while reference herein is made to a conventional diet of corn and soy, the disclosed compound may also be used to advantage in combination with other forms of conventional animal feed, such as, but not limited to, wheat.

Kinomics analysis of tissues collected from sacrificed birds fed the dietary mixture of the inventive compound and conventional feed suggests that the biomass causes an alteration of signaling in multiple growth-related pathways. These pathways include, but are not limited to, those associated with the activator protein 1 (AP-1), the vascular endothelial growth factor (VEGF), the mitogen-activated protein kinase (MAPK or MAP kinase), Ak strain transforming (Akt), and the neurotrophic tropomyosin-related kinase (NTRK). Evidence supports the conclusion that this alteration represents the activation of the various pathways.

The disclosed inventive concept has numerous advantageous applications in humans and animals including but not limited to: (1) improving growth rate without the use of further supplements such as antibiotics, enzymes, probiotics, or antimicrobials and (2) providing an all-natural solution to the need for improved growth rate. The disclosed compound may also have growth-enhancing effects when fed to bovine, porcine, avian, equine, ovine, leporidae, and caprine species.

### DESCRIPTION OF THE DRAWINGS

For a more complete understanding of this invention, reference should now be made to the accompanying figures which are described as follows:

FIG. 1 is a bar graph illustrating changes in average body weight for animal subjects between Days 1, 14, 21, 28, 35, and 42 for both animals fed a control diet and those fed a diet supplemented with the disclosed inventive composition;

FIG. 2 is a bar graph illustrating changes in average feed intake for animal subjects between Days 1, 14, 21, 28, 35, and 42 for both animals fed a control diet and those fed a diet supplemented with the disclosed inventive composition;

FIG. 3 is a bar graph illustrating changes in average corrected feed conversion ratios (FCRs) for animal subjects between Days 1, 14, 21, 28, 35, and 42 for both animals fed a control diet and those fed a diet supplemented with the disclosed inventive composition; and

FIG. 4 is a bar graph illustrating changes in breast meat yield for animal subjects between Days 1, 14, 21, 28, 35, and 42 for both animals fed a control diet and those fed a diet supplemented with the disclosed inventive composition.

### DETAILED DESCRIPTION OF THE PREFERRED EMBODIMENT

In the following description, various operating parameters and components are described for different constructed embodiments. These specific parameters and components are included as examples and are not meant to be limiting. Unless otherwise noted, all technical and scientific terms used herein are to be accorded their common meanings as would be understood by one having ordinary skill in the art.

The method of the disclosed inventive concept proposes the use of a compound comprising an algal biomass as well as related materials including, for example, algal supernatant, symbiont bacteria, bacterial biomass, and bacterial fermentate. The inventive compound is combined with conventional feed to create a feed mixture that is fed to chickens, for example, broiler chickens, as well as other animals, to improve growth efficiency of the birds.

### The Compound Used in Growth Promotion Method and Treatment

The disclosed growth promotion method and treatment utilizes an effective compound comprising an algal biomass and related materials. By administering the compound early in broiler life, optimal growth rate via immune modulation can be accomplished. The effective compound may be derived from a lipopolysaccharide (LPS) of a gram-negative bacteria or may be derived from a source other than a lipopolysaccharide.

As used herein, “modulator” refers to an activator, an inhibitor, or both. Modulation may be the result of activity by at least one Toll-like receptor (TLR), such as TLR4 or possibly TLR2. As used herein, the term “inhibitor” refers to a molecule that reduces or attenuates the activity induced by another molecule. By way of example, a compound that might block the LPS-dependent activation of TLRs present on the surface of immune cells in humans and animals would be regarded as an inhibitor of this particular pathway.

As used herein, the term “algal culture” is defined as an algal organism and bacteria (one or more types) that grow together in a liquid medium. Unless expressly stated otherwise, the term “algal biomass” refers to the algal cells and bacterial cells (with the liquid culture medium removed). The “algal biomass” can be wet material or dried material.

Unless expressly stated otherwise, the term “algal supernatant” is defined as the culture medium in which the algal biomass is grown that contains excreted compounds from the algal biomass. Algal supernatant is obtained by growing algal biomass in culture medium for an appropriate length of time and then removing the algal and bacterial cells by filtration and/or centrifugation.

It is known that bacteria that are part of the Variovorax genus and the Rhodobacter genus are metabolically versatile. Variovorax is a gram-negative aerobic bacterium that can grow under a variety of conditions. It is part of the subclass Proteobacteria and is capable of metabolically utilizing several natural compounds generated by plants. Rhodobacter can grow under a broad variety of conditions, including both photosynthesis and chemosynthesis. Growth can also be achieved under both anaerobic and aerobic conditions. Rhodobacter sphaeroides represent a gram-negative facultative bacterium and is a member of the α-3 subdivision of the Proteobacteria.

Embodiments of the compound used in the growth promotion method and treatment as set forth herein include one or more compounds produced by gram-negative bacterial strains for use as selective modulators of various TLR-related signaling pathways. The disclosed inventive concept involves any combination of three fundamental steps: (1) the gram-negative bacteria produces LPS/Lipid A compounds; (2) the LPS/Lipid compounds modulate TLR pathways; and (3) downstream effects result in enhanced innate and adaptive immune processes, thereby preventing or reversing the growth-inhibiting effects of diseases, such as coccidiosis.

In an embodiment, the compounds used as selective modulators of the TLR signaling pathways are produced from a Variovorax paradoxus strain. The Variovorax paradoxus strain may be a naturally occurring strain found in an algal biomass and/or algal supernatant products. For example, the algal biomass may comprise the algal species Klebsormidium flaccidum. More specifically, the algal biomass culture may comprise the algal strain Klebsormidium flaccidum, var. ZIVO.

In another embodiment, the compounds used as selective modulators of TLR signaling pathways are produced from a Rhodobacter sphaeroides strain. Extensive studies have been undertaken generally regarding the structure and function of Rhodobacter sphaeroides. More focused studies have examined the photosynthetic characteristics of Rhodobacter sphaeroides. While it is known that lipopolysaccharides from Rhodobacter sphaeroides are effective TLR4 antagonists in human cells, which prevent TLR4-mediated inflammation by means of blocking LPS/TLR4 signaling, the inventors had uncovered an LPS compound derived from Rhodobacter sphaeroides that proved effective as a coccidiostat in poultry. While initial data suggested inhibition by an LPS-like molecule, it was not until specific testing directed toward Rhodobacter sphaeroides revealed the effectiveness of this bacterial product in the treatment of disease, such as in the treatment of coccidiosis in poultry. Research further showed that combining a bacterial product shown to act as a TLR4 inhibitor in some species for example, in human cells, and possibly in poultry cells as well as in others) with an activator of TLR2 (such as LPS from many gram-negative bacteria) provides enhanced growth promotion in animals.

Accordingly, embodiments of the compound used according to the present disclosure are directed to one or more compounds produced by a gram-negative bacterial strain of the genus Variovorax or the genus Rhodobacter for use as selective modulators of TLR signaling pathways. A specific embodiment of the disclosed inventive concept is directed to the use of an LPS/Lipid A compound used as a selective modulator of TLR signaling pathways produced from a Variovorax strain and the Rhodobacter sphaeroides strain.

The LPS/Lipid A compound employed herein may be obtained from the Variovorax strain or the Rhodobacter sphaeroides strain by any suitable method, but in specific embodiments they are extracted using standard multi-step LPS extraction protocols, such as: (1) extracting freeze-dried bacteria with a solution of phenol/guanidine thiocyanate and collecting the water layer for freeze-drying; (2) resolubilizing the freeze-dried fraction in water; (3) ultrafiltration of the solubilized fraction to remove low molecular weight substances and salts; (4) affinity purifying the high-molecular weight fraction using a polymyxin B resin column such as Affi-prep polymyxin matrix material (Bio-Rad), from which an active fraction is eluted with 1 deoxycholate and, optionally; (5) performing additional purification using size-exclusion chromatography.

In some examples, multiple types of LPS extraction protocols are employed to obtain an LPS compound from the bacteria, and extraction procedures may be performed more than once. Once the LPS compound is extracted and purified from the bacteria, the Lipid A fraction may be prepared by acid hydrolysis or other suitable technique.

The one or more LPS/Lipid A compounds derived from gram-negative bacterial strains, such as Variovorax or Rhodobacter sphaeroides, may selectively modulate the TLR signaling pathways by activating or inhibiting to improve immune health in a variety of uses and applications. In an embodiment, the LPS/Lipid A compound derived from Variovorax or Rhodobacter sphaeroides may be incorporated within an algal-based feed ingredient to improve gut health of poultry.

The disclosed LPS/Lipid A compound derived from Variovorax or Rhodobacter sphaeroides may be used to improve the health of poultry through a variety of mechanisms. For example, the LPS/Lipid A compound may protect against internal inflammation in poultry by negatively regulating inflammatory mediators via the down-regulation of TLR4 expression and the inhibition of NF-kappa B activation. In another example, the LPS/Lipid A compound may inhibit the activation of TLR4 in poultry by interfering with cysteine residue-mediated receptor dimerization. In yet another example, the LPS/Lipid A compound may inhibit the ability of non-infectious and infectious stimuli to interact with TLR4 and trigger a pro-inflammatory response to improve poultry gut integrity. Research further showed that combining a TLR4 inhibitor with an activator of TLR2 (such as LPS from many gram-negative bacteria) provides disease preventing or disease treatment effect.

### Growth Promotion Method and Treatment

Non-limiting examples of methods for promoting animal growth are set forth. It is to be understood that while the following methods are directed to the enhancement of growth in poultry, the disclosed methods may apply as well to other animals as well as humans. According, the described growth promotion methods and treatments are not intended as being solely for use in poultry.

The growth promoting benefits of the disclosed inventive concept may be obtained by including the active agents in natural animal feed in a number of different forms including biomass or supernatant from algal cultures, biomass consisting of certain gram-negative bacteria such as bacteria from the genus Rhodobacter or Variovorax, or as LPS derived from gram negative bacteria including but not limited to the genus Rhodobacter or Variovorax. In all instances the combined conventional feed and disclosed inventive composition is fed to the animals preferably beginning Day 01.

When including the disclosed inventive concept as algal biomass in animal feed, the combined batch is preferably provided in an amount of between about 0.5 lbs. composition per ton of finished feed to about 11.0 lbs. composition per ton of finished feed, is more preferably provided in an amount of between about 1.0 lbs. composition per ton of finished feed to about 5.0 lbs. composition per ton of finished feed, and is most preferably provided in an amount of between about 3.0 lbs. composition per ton of finished feed to about 4.0 lbs. composition per ton of finished feed. The ideal suggested and non-limiting ratio is about 3.5 lbs. composition per ton of finished feed for maximum effect.

When including the disclosed inventive concept as bacterial biomass in animal feed, the combined batch is preferably provided in an amount of between about 20.0 g composition to ton of finished feed to about 250.0 g composition to ton of finished feed, is more preferably provided in an amount of between about 125.0 g composition to ton finished feed to about 175.0 g composition to ton of finished feed, and is most preferably provided in an amount of between about 100.0 g composition per ton of finished feed to about 150.0 g composition per ton of finished feed. The ideal suggested and non-limiting ratio is about 125.0 g composition per ton of finished feed for maximum effect.

When including the disclosed inventive concept as LPS added to animal feed, the combined batch is preferably provided in an amount of between about 0.1 g composition to ton of finished feed to about 10.0 g composition to ton of finished feed, is more preferably provided in an amount of between about 0.5 g composition to ton finished feed to about 7.5 g composition to ton of finished feed, and is most preferably provided in an amount of between about 1.0 g composition per ton of finished feed to about 5.0 g composition per ton of finished feed. The ideal suggested and non-limiting ratio is about 2.5 g composition per ton of finished feed for maximum effect.

### Research Results

Two studies were undertaken to determine the effect of the disclosed inventive composition on animal growth when administered as part of a diet for subject animals. According to the first study, the growth promotion method and treatment according to the present disclosed inventive concept was tested at a research university in a 42-day broiler pen study. Overall, the results showed that birds fed a feed composition including the disclosed inventive compound and a corn/soy mixture demonstrated higher Body Weight Gain (BWG) and a lower Feed Conversion Ratio (FCR) compared to control animals fed only the corn/soy mixture. According to the second study conducted at an agricultural contract research organization, it was demonstrated that healthy birds fed a composition containing the inventive compound and raised under conditions that included environmental exposure to pathogens had increased body weight, lower FCR, and increased breast muscle tissue compared to birds fed the traditional diet without the inventive compound. These studies demonstrate the positive effects of the inventive compound when combined with conventional animal feed.

### First Study

According to the present, non-limiting example, the inventive compound is defined as the algal biomass as set forth above and related materials including algal supernatant and symbiont bacteria. In the first study, the inventive compound was mixed with conventional feed to form a supplemented “feed mixture” at a fixed ratio. This ratio was maintained throughout the test period. The bird flock was divided into a control group fed only conventional corn-soy feed and an experimental group fed the supplemented feed mixture.

Performance results from the first study are set forth in the following tables. As is conventionally applied, “BW” refers to Body Weight, “BWG” refers to Body Weight Gain, “FI” refers to Feed Intake, and “FCR” refers to Feed Conversion Ratio.

TABLE 1
Performance of healthy Ross 308 broilers fed basal diet ±
inventive biomass ingredient for 42 d divided into 14-d starter,
grower, and finisher periods on a pen basis

| | Corn-Soy Control | Corn-Soy + Biomass |
|---|---|---|
| d0 BW, kg | 0.6240 | 0.6203 |
| Starter (d0-d14) | | |
| BWG, kg | 4.2113 | 4.3615 |
| FI, kg | 5.5238 | 5.7440 |
| FCR | 1.3212 | 1.3341 |
| Grower (d14-d28) | | |
| BWG, kg | 11.7450 | 12.1718 |
| FI, kg | 18.9170 | 19.2858 |
| FCR | 1.6187 | 1.5730 |
| Finisher (d28-d42) | | |
| BWG, kg | 17.5465 | 19.2838 |
| FI, kg | 31.4620 | 32.7553 |
| FCR | 1.8000 | 1.6873 |
| Overall (d0-d42) | | |
| BWG, kg | 33.5028 | 35.8170 |
| FI, kg | 55.9028 | 57.7850 |
| FCR | 1.6581 | 1.6042 |

TABLE 2
Weekly performance of healthy Ross 308 broilers fed basal
diet ± inventive biomass ingredient for 42 d on a pen basis

| | Corn-Soy Control | Corn-Soy + Biomass |
|---|---|---|
| d0 BW, kg | 0.6240 | 0.6203 |
| Week 1 | | |
| BW, kg | 1.9600 | 1.9795 |
| BWG, kg | 1.3360 | 1.3592 |
| FI, kg | 1.5273 | 1.5613 |
| FCR | 1.1459 | 1.1494 |
| Week 2 | | |
| BW, kg | 4.8353 | 4.9818 |
| BWG, kg | 2.8753 | 3.0023 |
| FI, kg | 3.9965 | 4.1828 |
| FCR | 1.4221 | 1.4224 |
| Week 3 | | |
| BW, kg | 9.7667 | 10.0803 |
| BWG, kg | 4.9315 | 5.0985 |
| FI, kg | 7.3555 | 7.5520 |
| FCR | 1.5049 | 1.4664 |
| Week 4 | | |
| BW, kg | 16.5803 | 17.1535 |
| BWG, kg | 6.8135 | 7.0733 |
| FI, kg | 11.5615 | 11.7338 |
| FCR | 1.6668 | 1.6627 |
| Week 5 | | |
| BW, kg | 25.1950 | 26.5713 |
| BWG, kg | 8.6148 | 9.4178 |
| FI, kg | 14.5903 | 15.1878 |
| FCR | 1.6657 | 1.6011 |
| Week 6 | | |
| BW, kg | 34.1268 | 36.4373 |
| BWG, kg | 8.9318 | 9.8660 |
| FI, kg | 16.8718 | 17.5675 |
| FCR | 1.8729 | 1.7920 |

TABLE 3
Performance of healthy Ross 308 broilers fed basal diet ±
inventive biomass ingredient for 42 d divided into 14-d starter,
grower, and finisher periods on a per bird basis

| | Corn-Soy Control | Corn-Soy + Biomass |
|---|---|---|
| d0 BW, kg | 0.0446 | 0.0443 |
| Starter (d0-d14) | | |
| BWG, kg | 0.3059 | 0.3127 |
| FI, kg | 0.4017 | 0.4119 |
| FCR | 1.3297 | 1.3341 |
| Grower (d14-d28) | | |
| BWG, kg | 0.8997 | 0.9145 |
| FI, kg | 1.4531 | 1.4496 |
| FCR | 1.6191 | 1.5889 |
| Finisher (d28-d42) | | |
| BWG, kg | 1.4279 | 1.5116 |
| FI, kg | 2.5608 | 2.5685 |
| FCR | 1.8040 | 1.6873 |
| Overall (d0-d42) | | |
| BWG, kg | 2.7249 | 2.8083 |
| FI, kg | 4.5511 | 4.5326 |
| FCR | 1.6581 | 1.6042 |

TABLE 4
Weekly performance of healthy Ross 308 broilers fed basal diet ±
inventive biomass ingredient for 42 d on a per bird basis

| | Corn-Soy Control | Corn-Soy + Biomass |
|---|---|---|
| d0 BW, kg | 0.0446 | 0.0443 |
| Week 1 | | |
| BW, kg | 0.1405 | 0.1414 |
| BWG, kg | 0.0958 | 0.0971 |
| FI, kg | 0.1095 | 0.1115 |
| FCR | 1.1459 | 1.1494 |
| Week 2 | | |
| BW, kg | 0.3514 | 0.3572 |
| BWG, kg | 0.2088 | 0.2152 |
| FI, kg | 0.2906 | 0.2999 |
| FCR | 1.4221 | 1.4224 |
| Week 3 | | |
| BW, kg | 0.7426 | 0.7581 |
| BWG, kg | 0.3739 | 0.3891 |
| FI, kg | 0.5592 | 0.5679 |
| FCR | 1.5049 | 1.4664 |
| Week 4 | | |
| BW, kg | 1.2713 | 1.2898 |
| BWG, kg | 0.5227 | 0.5317 |
| FI, kg | 0.8753 | 0.8817 |
| FCR | 1.6668 | 1.6627 |
| Week 5 | | |
| BW, kg | 2.0483 | 2.0833 |
| BWG, kg | 0.7003 | 0.7379 |
| FI, kg | 1.1857 | 1.1909 |
| FCR | 1.6681 | 1.6011 |
| Week 6 | | |
| BW, kg | 2.7758 | 2.8570 |
| BWG, kg | 0.7276 | 0.7737 |
| FI, kg | 1.3584 | 1.3776 |
| FCR | 1.8729 | 1.7920 |

### First Study—Analysis Methodology

As set forth in the tables, the birds of the control group and the experimental group were weighed at various times during the study. Data were collected based on both growth stage (starter, grower, and finisher) and on a 42 day/six-week basis. For the growth stage analysis, the birds of each group were first weighed prior to treatment, then at each of the starter growth stage (d0-d14), the grower growth stage (d14-d28), and the finisher growth stage (d28-d42). Overall (d0-d42) results are also highlighted.

### First Study—Results

In general, analysis of the results supports the conclusion that inclusion of the innovative compound as part of a convention diet leads to a significant increase in growth and weight gain when compared to the control flock, particularly during the finisher stage of growth. The weight gains between d0 and d14 (starter) and again between d14-d28 (grower) of the experimental group over the control group are in the range of between 3.57% and 3.63%. However, the weight gain between d28 and d42 (finisher) are a significant 9.9%, thereby demonstrating that the addition of the inventive compound to conventional feed produces significant positive results, particularly at the later stages of bird growth.

Moreover, following treatment with the disclosed compound on selected birds, samples of both treated and non-treated birds were examined by gross necropsy which included internal examination. Kinomic analysis of tissues collected from sacrificed birds fed the dietary mixture of the inventive compound and conventional feed confirmed that the biomass alters multiple growth-related pathways as proposed, thus initiating pathway activation.

### Second Study

According to the second study, broiler chickens were fed a conventionally formulated corn/soybean meal diet with or without the inventive compound and raised to 42 days of age in pens containing built-up litter from a minimum of 3-previous flocks to simulate the typical health and growth stressors experienced in poultry production in the U.S.

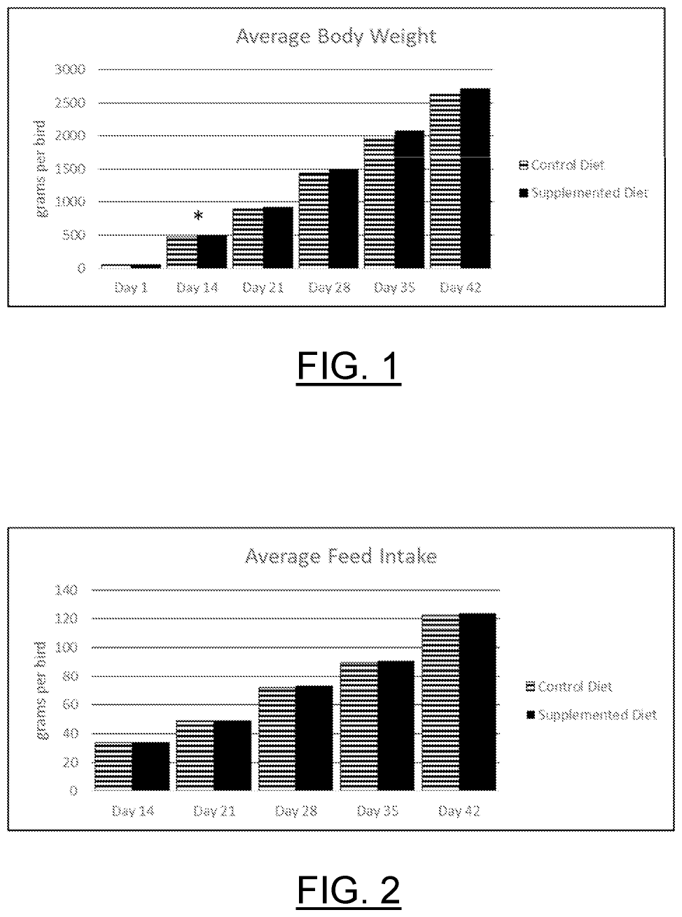

Performance results from the second study are set forth in the following table, the results of which are summarized in the accompanying FIGS. 1-4. Statistically significant results are highlighted in bold in the table and denoted by asterisks above selected bars in the figures.

TABLE 5
Illustrates changes in average body weight, feed intake, corrected feed conversion ratios, and
breast meat yield for animal subjects between Days 1, 14, 21, 28, 35, and 42 for both animals
fed a control diet and those fed a diet supplemented with the disclosed inventive composition.

| | Control Diet | Supplemented Diet | P Value |
|---|---|---|---|
| | Average Body Weight (g per bird) | | |
| Day 1 | 57.47 | 57.07 | 0.175 |
| Day 14 | 478.92 | 487.72 | 0.009 |
| Day 21 | 896.10 | 926.20 | 0.006 |
| Day 28 | 1438.40 | 1491.30 | 0.006 |
| Day 35 | 2001.70 | 2079.60 | 0.006 |
| Day 42 | 2629.40 | 2719.80 | 0.003 |
| | Average Feed Intake (g per bird per day) | | |
| Day 14 | 34.03 | 33.91 | 0.832 |
| Day 21 | 49.03 | 49.24 | 0.812 |
| Day 28 | 72.11 | 73.20 | 0.370 |
| Day 35 | 89.17 | 90.97 | 0.320 |
| Day 42 | 122.25 | 123.92 | 0.442 |
| | FCR Corrected | | |
| Days 1-14 | 1.129 | 1.101 | 0.022 |
| Days 1-21 | 1.223 | 1.187 | 0.002 |
| Days 1-28 | 1.353 | 1.323 | 0.041 |
| Days 1-35 | 1.514 | 1.485 | 0.116 |
| Days 1-42 | 1.737 | 1.698 | 0.105 |
| | Breast Meat Yield | | |
| Carcass Yield (not chilled, % of WOG) | 75.25 | 75.27 | 0.974 |
| Carcass Yield (chilled) | 78.26 | 78.24 | 0.965 |
| Pectoralis Minor Breast Yield | 97.91 | 102.85 | 0.018 |
| Pectoralis Major Breast Yield | 376.68 | 395.69 | 0.015 |
| Total Breast Yield | 474.59 | 498.54 | 0.015 |
| Pec Major Breast Yield (% live weight) | 18.10 | 18.02 | 0.322 |
| Pec Major Breast Yield (% pre-chill weight) | 24.16 | 24.02 | 0.422 |
| Pec Major Breast Yield (% post-chill weight) | 23.22 | 23.10 | 0.466 |
| Pec Minor Breast Yield (% tenders live weight) | 4.71 | 4.68 | 0.372 |
| Pec Minor Breast Yield (% tenders pre-chill) | 6.28 | 6.24 | 0.492 |
| Pec Minor Breast Yield (% tenders post-chill) | 6.04 | 6.00 | 0.518 |
| Total Deboned Breast Yield (% live weight) | 22.80 | 22.70 | 0.306 |
| Total Deboned Breast Yield (% pre-chill) | 30.43 | 30.26 | 0.427 |
| Total Deboned Breast Yield (% post-chill) | 29.25 | 29.10 | 0.476 |

### Second Study—Analysis Methodology

Birds were evaluated in terms of physical live performance, including measurements at days 14, 21, 28, 35, and 42 for body weight and feed intake, which were used to calculate the feed conversion ratio (FCR) across the same time periods. At 42 days of age, 10 birds per pen (n=120 birds per group) were processed to assess carcass and meat quality.

### Second Study—Results

The results of the second study are set forth in Table 5 and in FIGS. 1-4.

As illustrated in Table 5 and in FIG. 1, birds receiving the diet supplemented with the compound according to the disclosed inventive concept had numerically higher body weight throughout the entire study compared to the control group consuming feed without the inventive compound. Also as illustrated in Table 5 and FIG. 1, Body weight differences between groups reached statistical significance at 14 days of age and continued to be significantly higher at 21, 28, 35, and 42 days of age. Despite the differences in body weight, there were no significant differences in feed intake for the two groups as illustrated in Table 5 and FIG. 2. This finding indicates that the birds consuming the supplemented diet converted feed into body mass more efficiently than the birds fed the control diet.

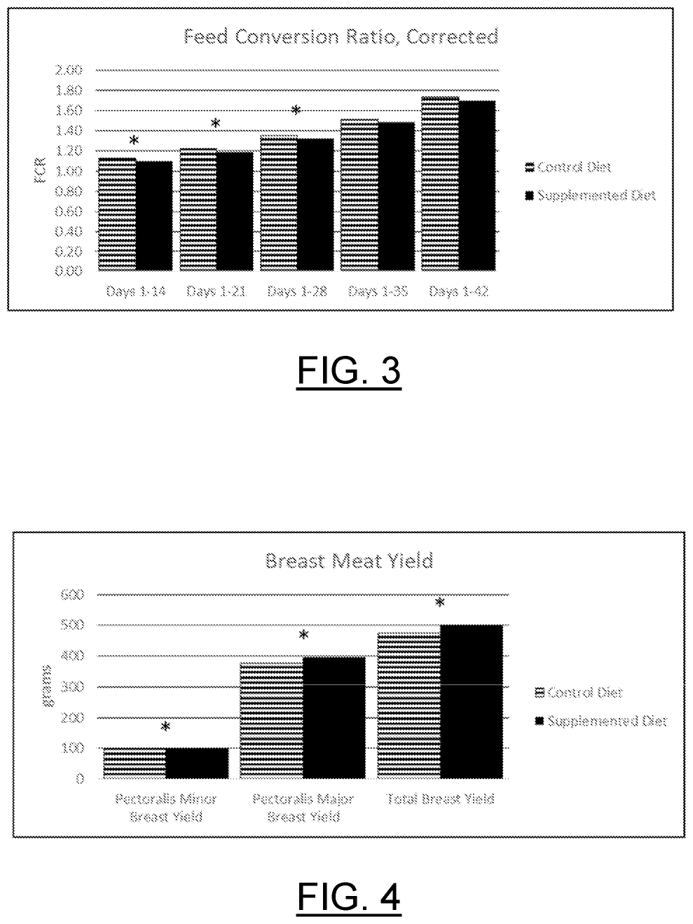

As illustrated in Table 5 and in FIG. 3, birds receiving the diet supplemented with the inventive compound had numerically lower FCR throughout the entire study duration compared to the control group. FCR differences between groups reached statistical significance (P<0.05) for the periods spanning Days 1-14, 1-21, and 1-28.

Perhaps most significantly, while there were no differences in overall carcass yield as illustrated in Table 5 and in FIG. 4, the total breast weight, as well as the Pectoralis major (breast fillets) and Pectoralis minor (tender) weights, were increased in the group fed the diet supplemented with the inventive compound disclosed herein.

What is claimed is:

1. A method for promoting the growth of an animal through the use of a modified natural diet, the method comprising feeding to the animal an effective amount of a composition derived from the group consisting of a biomass or a supernatant from an algal culture, a biomass consisting of Gram-negative bacteria, and a lipopolysaccharide derived from Gram-negative bacteria, the method including the step of initially feeding the animal the effective amount of the composition beginning in the first week of life.

2. The method of claim 1, whereby the composition is mixed with a feed ration portion prior to feeding the animal.

3. The method of claim 2, where the Gram-negative bacteria is from the genus Rhodobacter or Variovorax.

4. The method of claim 2, wherein the composition derived from the biomass or the supernatant is fed to the animal in an amount providing from about 0.5 lbs. composition per ton of finished feed to about 11.0 lbs. composition per ton of finished feed.

5. The method of claim 2, wherein the composition derived from the biomass or the supernatant is fed to the animal in an amount providing from about 1.0 lbs. composition per ton of finished feed to about 5.0 lbs. composition per ton of finished feed.

6. The method of claim 2, wherein the composition derived from the biomass or the supernatant is fed to the animal in an amount providing from about 3.0 lbs. composition per ton of finished feed to about 4.0 lbs. composition per ton of finished feed.

7. The method of claim 2, wherein the composition derived from the biomass consisting of Gram-negative bacteria is fed to the animal in an amount providing from about 20.0 g composition per ton of finished feed to about 250.0 g composition per ton of finished feed.

8. The method of claim 2, wherein the composition derived from the biomass consisting of Gram-negative bacteria is fed to the animal in an amount providing from about 125.0 g composition per ton of finished feed to about 175.0 g composition per ton of finished feed.

9. The method of claim 2, wherein the composition derived from the biomass consisting of Gram-negative bacteria is fed to the animal in an amount providing from about 100.0 g composition per ton of finished feed to about 150.0 g composition per ton of finished feed.

10. The method of claim 2, wherein the composition comprising the lipopolysaccharide derived from Gram-negative bacteria is fed to the animal in an amount providing from about 0.1 g composition per ton of finished feed to about 10.0 g composition per ton of finished feed.

11. The method of claim 2, wherein the composition comprising the lipopolysaccharide derived from Gram-negative bacteria is fed to the animal in an amount providing from about 0.5 g composition per ton of finished feed to about 7.5 g composition per ton of finished feed.

12. The method of claim 2, wherein the composition comprising the lipopolysaccharide derived from Gram-negative bacteria is fed to the animal in an amount providing from about 1.0 g composition per ton of finished feed to about 5.0 g composition per ton of finished feed.

13. The method of claim 1 wherein the composition comprising the lipopolysaccharide derived from Gram-negative bacteria composition is adapted for growth enhancement in poultry.

14. The method of claim 1, wherein the composition is formulated for feeding to bovine, porcine, avian, equine, ovine, leporidae, and caprine species.

15. A method for promoting the growth of an animal, the method comprising feeding to the animal an effective amount of a composition derived from a biomass or a lipopolysaccharide derived from Gram-negative bacteria, the method including the step of feeding the animal the effective amount of the composition beginning with the first week of life, whereby growth promotion is achieved based on the combined feed and composition diet alone and without the need for growth promoting hormones or antibiotics.

16. The method of claim 15, wherein the biomass is selected from the group consisting of an algal biomass and a bacterial biomass,

17. The method of claim 15, wherein the composition is formulated for feeding to bovine, porcine, avian, equine, ovine, leporidae, and caprine species.

18. The method of claim 15, wherein said Gram-negative bacteria is a member of the group Variovorax.

19. A composition for growth promotion in animals, the composition comprising effective amounts of a feed ingredient is a biomass selected from the group consisting of an algal biomass, a bacterial biomass, and a lipopolysaccharide derived from Gram-negative bacteria.

20. The composition of claim 19 wherein the bacterial biomass includes one of a supernatant, a symbiont bacteria, or bacterial fermentate.
